(19) United States
(12) Patent Application Publication
(10) Pub. No.: US 2022/0008257 A1
(43) Pub. Date: Jan. 13, 2022

(54) Anally Insertable Absorptive Device

(71) Applicant: Blessing Ladomi, Los Angeles, CA (US)

(72) Inventor: Blessing Ladomi, Los Angeles, CA (US)

(21) Appl. No.: 16/923,665

(22) Filed: Jul. 8, 2020

(51) Int. Cl. A61F 13/20; A61F 2/00; A61F 13/34

(52) U.S. Cl. CPC A61F 13/2011; A61F 2/0022; A61F 13/34; A61F 2013/842; A61F 13/2051; A61F 13/2074; A61F 13/2022

(57) ABSTRACT

An anally insertable absorptive device for minimizing anal leakage includes a pad that is substantially cylindrically shaped and configured to be inserted through an anus into a rectum of a user. The pad is absorptive and is configured to absorb liquid in the rectum to prevent anal leakage. A string is coupled to and extends from a trailing end of the pad. The string is configured to be grasped in digits of a hand of the user, positioning the user to pull on the string to extract the pad from the rectum through the anus.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to absorptive devices and more particularly pertains to a new absorptive device for minimizing anal leakage.

### (2) Description of Related Art Including Information Disclosed Under 37 CFR 1.97 and 1.98

The prior art relates to absorptive devices.

### BRIEF SUMMARY OF THE INVENTION

An embodiment of the disclosure meets the needs presented above by generally comprising a pad that is substantially cylindrically shaped and configured to be inserted through an anus into a rectum of a user. The pad is absorptive and is configured to absorb liquid in the rectum to prevent anal leakage. A string is coupled to and extends from a trailing end of the pad. The string is configured to be grasped in digits of a hand of the user, positioning the user to pull on the string to extract the pad from the rectum through the anus.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of an anally insertable absorptive device according to an embodiment of the disclosure.

FIG. 2 is a back view of an embodiment of the disclosure.

FIG. 3 is a front view of an embodiment of the disclosure.

FIG. 4 is a side view of an embodiment of the disclosure.

FIG. 5 is an in-use view of an embodiment of the disclosure.

### DETAILED DESCRIPTION OF THE INVENTION

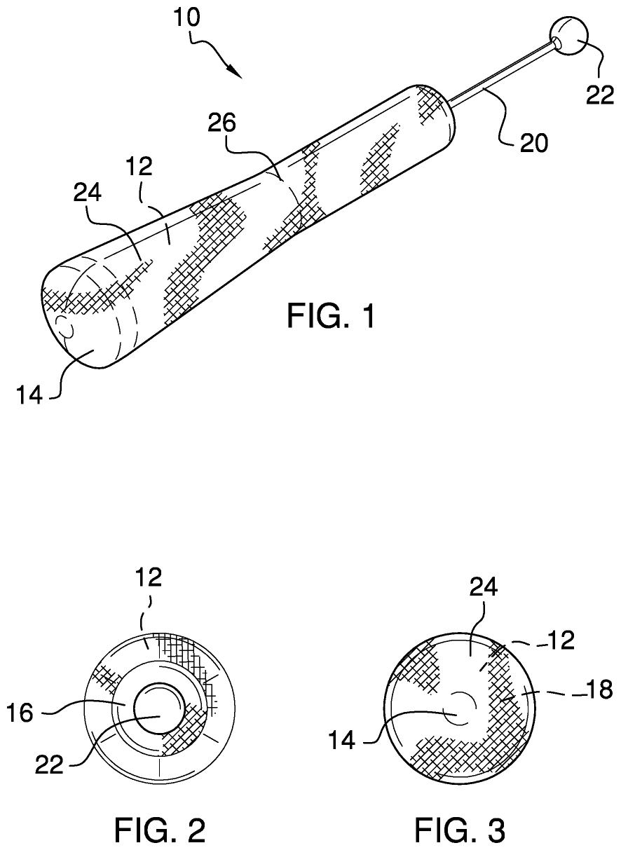

With reference now to the drawings, and in particular to FIGS. 1 through 5 thereof, a new absorptive device embodying the principles and concepts of an embodiment of the disclosure and generally designated by the reference numeral 10 will be described.

As best illustrated in FIGS. 1 through 5, the anally insertable absorptive device 10 generally comprises a pad 12 that is substantially cylindrically shaped and configured to be inserted through an anus into a rectum of a user. The pad 12 is absorptive and is configured to absorb liquid in the rectum to prevent anal leakage. The device 10 can be used to improve personal hygiene in a person suffering from soft stool or diarrhea and can help in the healing of a gluteal skin wound, such as a decubitus ulcer, by preventing fecal matter from contaminating the wound.

The pad 12 comprises at least one of cotton, rayon, wood pulp, cellulose, polypropylene fiber, polyethylene fiber, and superabsorbent polymer so that the pad 12 is configured to absorb liquid. The pad 12 has a leading end 14 and a trailing end 16. The leading end 14 is arcuate and thus is configured to facilitate insertion of the pad 12 through the anus into the rectum. The trailing end 16 is arcuate and thus is configured to facilitate extraction of the pad 12 from the rectum via the anus.

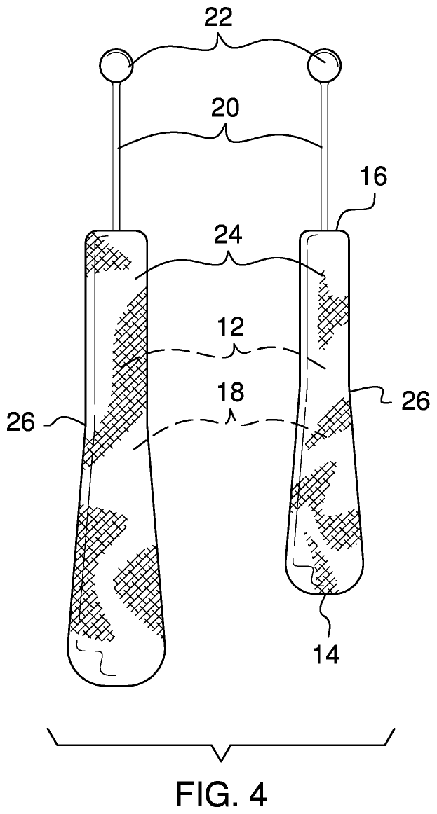

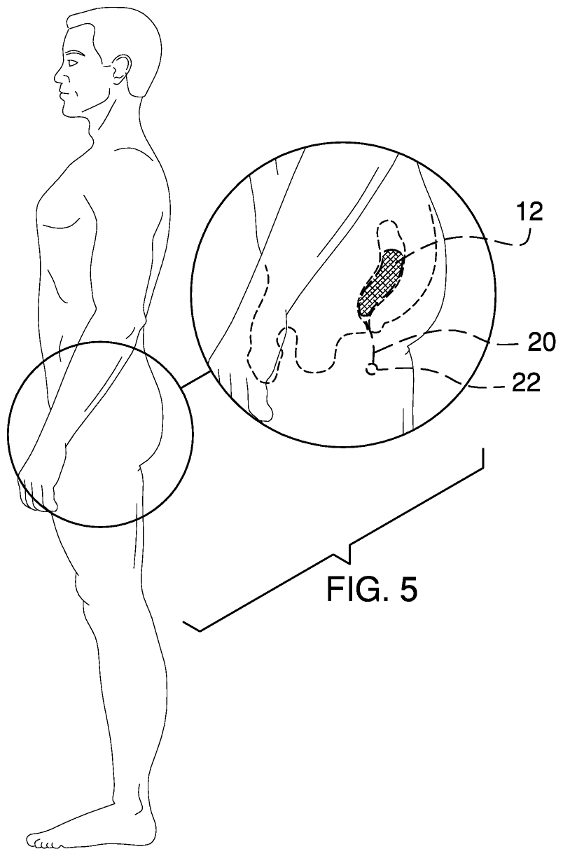

The leading end 14 may be circumferentially larger than the trailing end 16, as shown in FIG. 4. The pad 12 may taper from the leading end 14 to proximate to a midpoint 26 of the pad 12, as shown in FIG. 4. The pad 12 being tapered as such is easier to extract from the rectum through the anus.

The pad 12 may comprise at least one of activated charcoal, silica, and zeolite so that the pad 12 is configured to adsorb gas in the rectum. The pad 12 also may be permeated with an aroma compound 18 so that the pad 12 is configured to release the aroma compound 18 into gas that passes through the anus. The pad 12 is sized so that the pad 12 is substantially complementary to a volume of the rectum. Thus, the pad 12 may be large sized and small sized, as shown in FIG. 4.

A string 20 is coupled to and extends from a trailing end 16 of the pad 12. The string 20 is configured to be grasped in digits of a hand of the user, positioning the user to pull on the string 20 to extract the pad 12 from the rectum through the anus. A bead 22 that is coupled to the string 20 distal from the pad 12 is configured to deter slippage of the digits of the hand grasping the string 20. The bead 22 may be substantially spherically shaped, or may be alternatively shaped, such as disc shaped, egg shaped, and the like.

A shell 24 that is positioned around the pad 12 is permeable to water. The shell 24 is configured to facilitate insertion and extraction of the pad 12. The shell 24 also is configured to allow passage of liquid from the rectum into the pad 12. The shell 24 comprises at least one of polyethylene and polypropylene.

In use, the pad 12 is inserted through the anus into the rectum where it is positioned to absorb liquid to minimize anal leakage. The pad 12 also is configured to adsorb gas, helping to minimize flatulence. Additionally, the aroma compound 18 is released from the pad 12 into the gas in the rectum so that flatulence, should it occur, would be masked by the aroma compound 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. An anally insertable absorptive device comprising:
a pad, the pad being substantially cylindrically shaped wherein the pad is configured for inserting through an anus into a rectum of a user, the pad being absorptive wherein the pad is configured for absorbing liquid in the rectum for preventing anal leakage; and
a string coupled to and extending from a trailing end of the pad wherein the string is configured for grasping in digits of a hand of the user positioning the user for pulling on the string for extracting the pad from the rectum through the anus.

2. The device of claim 1, further including the pad comprising at least one of cotton, rayon, wood pulp, cellulose, polypropylene fiber, polyethylene fiber, and superabsorbent polymer wherein the pad is configured for absorbing liquid.

3. The device of claim 1, further including the pad having a leading end, the leading end being arcuate wherein the leading end is configured for facilitating insertion of the pad through the anus into the rectum.

4. The device of claim 1, further including the trailing end being arcuate wherein the trailing end is configured for facilitating extraction of the pad from the rectum via the anus.

5. The device of claim 1, further including the pad comprising at least one of activated charcoal, silica, and zeolite wherein the pad is configured for adsorbing gas in the rectum.

6. The device of claim 1, further including the pad being permeated with an aroma compound wherein the pad is configured for releasing the aroma compound into gas passing through the anus.

7. The device of claim 1, further including the pad being sized such that the pad is substantially complementary to a volume of the rectum.

8. The device of claim 1, further including a bead coupled to the string distal from the pad wherein the bead is configured for deterring slippage of the digits of the hand grasping the string.

9. The device of claim 8, further including the bead being substantially spherically shaped.

10. The device of claim 1, further including a shell positioned around the pad, the shell being permeable to water wherein the shell is configured for facilitating insertion and extraction of the pad and wherein the shell is configured for passing of liquid from the rectum into the pad.

11. The device of claim 10, further including the shell comprising at least one of polyethylene and polypropylene.

12. The device of claim 1, further including a leading end of the pad being circumferentially larger than the trailing end.

13. The device of claim 1, further including the pad tapering from the leading end to proximate to a midpoint of the pad.

14. An anally insertable absorptive device comprising:
a pad, the pad being substantially cylindrically shaped wherein the pad is configured for inserting through an anus into a rectum of a user, the pad being absorptive wherein the pad is configured for absorbing liquid in the rectum for preventing anal leakage, the pad comprising at least one of cotton, rayon, wood pulp, cellulose, polypropylene fiber, polyethylene fiber, and superabsorbent polymer wherein the pad is configured for absorbing liquid, the pad having a leading end and a trailing end, the leading end being arcuate wherein the leading end is configured for facilitating insertion of the pad through the anus into the rectum, the trailing end being arcuate wherein the trailing end is configured for facilitating extraction of the pad from the rectum via the anus, the pad comprising at least one of activated charcoal, silica, and zeolite wherein the pad is configured for adsorbing gas in the rectum, the pad being permeated with an aroma compound wherein the pad is configured for releasing the aroma compound into gas passing through the anus, the pad being sized such that the pad is substantially complementary to a volume of the rectum, the leading end of the pad being circumferentially larger than the trailing end, the pad tapering from the leading end to proximate to a midpoint of the pad;
a string coupled to and extending from the trailing end wherein the string is configured for grasping in digits of a hand of the user positioning the user for pulling on the string for extracting the pad from the rectum through the anus;
a bead coupled to the string distal from the pad wherein the bead is configured for deterring slippage of the digits of the hand grasping the string, the bead being substantially spherically shaped; and
a shell positioned around the pad, the shell being permeable to water wherein the shell is configured for facilitating insertion and extraction of the pad and wherein the shell is configured for passing of liquid from the rectum into the pad, the shell comprising at least one of polyethylene and polypropylene.
